(19) United States
(12) Patent Application Publication
(10) Pub. No.: US 2012/0215002 A1
(43) Pub. Date: Aug. 23, 2012

(54) SYNTHESIS OF OPTICALLY ACTIVE INTERMEDIATE FOR THE PREPARATION OF MONTELUKAST

(76) Inventor: Laurent Lefort, Maastricht (NL)

(21) Appl. No.: 13/500,252

(22) PCT Filed: Oct. 5, 2010
(86) PCT No.: PCT/EP2010/064796

§ 371 Date: May 8, 2012

(30) Foreign Application Priority Data
Oct. 9, 2009 (EP) 09172619.0
Jan. 29, 2010 (EP) 10152133.4

(51) Int. Cl. C07D 215/18

(52) U.S. Cl. 546/180

(57) ABSTRACT

The present invention relates to the synthesis of optically active alcohols by means of enantioselective hydrogenation of ketones in biphasic systems. In particular the present invention relates to the synthesis of an optically active alcohol of general formula (1).

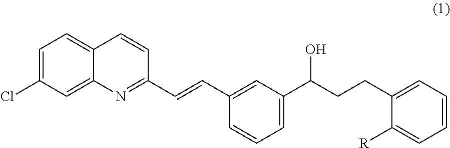

### FIELD OF THE INVENTION

The present invention relates to the synthesis of optically active alcohols by means of enantioselective hydrogenation of ketones in biphasic systems.

### BACKGROUND OF THE INVENTION

Enantioselective transfer hydrogenation of prochiral ketones was initially developed by Noyori et al. (J. Amer. Chem. Soc. 1995, 117, 7562) using ruthenium complexes with chiral mono-N-tosylated vicinal diamine ligands. Application of this technology is usually carried out in organic solvents with good yields and enantiomeric excess (ee). In view of this there was no immediate incentive to explore alternative solvents. Hence, only some approaches have been made to expand the Noyori technology to other systems, for instance by the application of water-soluble ligands. Unfortunately such ligands are often difficult to prepare. Alternatively, the use of surfactants has been reported (e.g. Adv. Synth. Catal. 2002, 344, 239) and also the application of aqueous micelles has been reported (J. Org. Chem. 2005, 70, 9424). Further expansion of the scope of enantioselective transfer hydrogenation would give access to optically active alcohols that are not available according to the methods of the present state of the art or which can only be prepared in relatively low yields and/or at relatively low ee-values. Hence, there remains a need for the development of new hydrogenation systems.

### DETAILED DESCRIPTION OF THE INVENTION

In the context of the present invention, the term ‘biphasic’ refers to a system comprising two phases wherein the two phases either are both in the liquid form and the two liquids are immiscible with respect to one another or wherein at least one of the phases is liquid and at least one of the phases is solid. Neither of the two phases is meant to encompass the substrate of the reaction that takes place in the biphasic system, although the substrate may be dissolved in one of the phases. Immiscible in this respect means a solubility of one liquid in the other of no more than 100 g.kg⁻¹ and preferably no more than 10 g.kg⁻¹. More preferably immiscible refers to a solubility of one liquid in the other of no more than 1 g.kg⁻¹.

The term ‘reduction’ refers to a process wherein hydrogen is added to a substrate, for example a process in which a ketone is converted to an alcohol.

It has surprisingly been found that metal complex catalyzed reductions can be carried out efficiently in biphasic systems. The preparation of a chiral intermediate for the anti-asthmatic drug montelukast is an example, but not a limitation, of the present invention.

The alcohol of general formula (1) is an intermediate in montelukast synthesis.

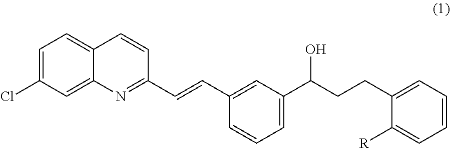

With R═—C(CH₃)₂OH and the molecule in the S-configuration, activation of the chiral hydroxyl group followed by displacement with a thiomethylcyclopropaneacetic acid moiety gives immediate access to montelukast.

In EP 480717 the synthesis of the R-form of (1) with R═—C(O)OCH₃ was described using BH₃ mediated reduction of a compound of formula (2) in the presence of a chiral catalyst.

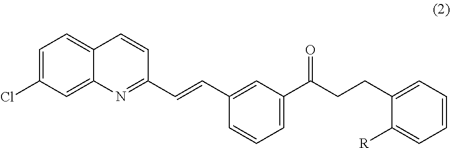

The same conversion was reported in U.S. Pat. No. 6,184,381, this time using the Noyori-catalyst chloro{[(1R,2R)-(−)-2-amino-1,2-diphenylethyl](4-toluenesulfonyl)amido}(mesitylene)ruthenium in tetrahydrofuran as solvent. Following a 72 h reaction the required alcohol was obtained in 68% yield and an ee of 92% of the R-isomer at a substrate to catalyst ratio of 200 only, which is not applicable to large scale production. In WO 2006/008562 the preparation of the S-form of (1) with R═—C(O)OCH₃ was described using a different ruthenium based catalyst in dimethylformamide as the solvent; here the yield was improved slightly to 75% and the ee of the S-form was 99.5% but an even lower substrate to catalyst ratio of 100 was used. In WO 2008/131932 a monophasic mixture of solvents was employed for asymmetric hydrogenation of (2) to give the S-form of (1) with R═—C(O)OCH₃ in 92% yield with an ee of 96.4% and a substrate to catalyst ratio of 482. A major drawback of the last development is that the use of hydrogen at high pressure is potentially dangerous and requires the use of special reactors. In addition, the individual components of monophasic solvent mixtures are difficult to recover and are therefore often discarded resulting in unwanted environmental burden and an increase in costs. Furthermore, still higher turnover number values are required in order to meet the specifications of the final product. Yet another drawback is the risk of reduction of the alkene moiety that is usually associated with high pressure hydrogenation.

In a first aspect the present invention provides a method for the preparation of an alcohol of general formula (1)

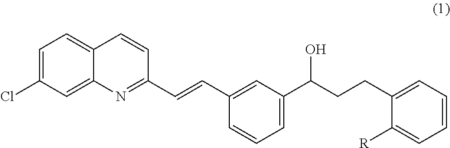

having a configuration of at least 60% R or at least 60% S and wherein R is —C(O)OR₁ or —C(CH₃)₂OR₂ with R₁ is hydrogen or a carboxylic acid protecting group and R₂ is hydrogen or an alcohol protecting group by reduction of a ketone of general formula (2)

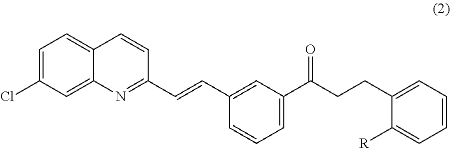

in the presence of a metal complex catalyst, characterized in that said reduction is carried out in a biphasic system.

Where R₁ is a carboxylic acid protecting group various groups are available to the skilled person. One of the preferred protecting groups is the methyl group (R₁═CH₃) but other suitable examples are allyl, 9-anthrylmethyl, benzyl, benzyloxymethyl, p-bromobenzyl, p-bromophenacyl, 3-buten-1-yl, n-butyl, sec-butyl, t-butyl, t-butyldimethylsilyl, di-t-butylmethylsilyl, t-butyldiphenylsilyl, cyclohexyl, carboxamidomethyl, cinnamyl, cyclopentyl, cyclopropylmethyl, 5-dibenzosuberyl, 2,6-dichlorobenzyl, 2,2-dichloro-1,1-difluoroethyl, 2,6-dimethoxybenzyl, 4-(dimethylaminocarbonyl)benzyl, 2,6-dimethylbenzyl, 1,1-dimethylpropyl, 1,2-dimethylpropyl, 2,2-dimethylpropyl, dimethylthiophosphinyl, 2-(9,10-dioxo)anthrylmethyl, diphenylmethyl, 2-(diphenylphosphino)ethyl, 1,3-dithianyl-2-methyl, ethyl, 9-fluorenylmethyl, 2-haloethyl, isobutyl, isopropyl, isopropyldimethylsilyl, p-methoxybenzyl, methoxyethoxymethyl, methoxymethyl, p-methoxyphenacyl, 1-methylbutyl, 2-methylbutyl, 3-methylbutyl, methyl carbonyl, α-methylcinnamyl, p-(methylmercapto)phenyl, α-methylphenacyl, 1-methyl-1-phenylethyl, 4-(methylsulfinyl)benzyl, methylthiomethyl, 2-methylthioethyl, o-nitrobenzyl, p-nitrobenzyl, bis(o-nitrophenyl)methyl, 2-(p-nitrophenylsulfenyl)ethyl), n-pentyl, phenacyl, phenyl, phenyldimethylsilyl, N-phthalimidomethyl, 4-picolyl, piperonyl, propyl, 1-pyrenylmethyl, 2-(2′-pyridyl)ethyl, 4-sulfobenzyl, 2-tetrahydrofuranyl, 2-tetrahydropyranyl, 2-(p-toluenesulfonyl)ethyl, 2,2,2-trichloroethyl, triethylsilyl, 2-(trifluoromethyl)-6-chromylmethyl, 2,4,6-trimethylbenzyl, 4-(trimethylsilyl)-2-buten-1-yl, trimethylsilyl, 2-(trimethylsilyl)ethyl, 2-(trimethylsilyl)ethoxymethyl and triphenylmethyl.

Where R₂ is an alcohol protecting group various methods for hydroxyl protection can be employed and are known to the person skilled in the art. As examples of classes of hydroxyl protecting groups R₂, mention can be made of ethers, acetals, esters, silyl groups and carbonates. For the purpose of the present invention, suitable groups R₂ are acetyl, allyl, 9-anthrylmethyl, benzoyl, benzyl, benzyl carbonyl, benzyloxymethyl, benzylsulfonyl, p-bromophenacyl, n-butyl, sec-butyl, t-butyl, t-butyldimethylsilyl, t-butyldiphenylsilyl, cyclohexyl, cyclopropylmethyl, 2,6-dichlorobenzyl, 2,2-dichloro-1,1-difluoroethyl, 4-(dimethylaminocarbonyl)benzyl, 2,6-dimethylbenzyl, dimethylphosphinyl, dimethylthiophosphinyl, ethyl, 9-fluorenecarboxyl, 2-formylbenzenesulfonyl, heptafluoro-p-tolyl, isobutyl, isopropyl, levulinyl, methanesulfonyl, p-methoxybenzyl, methoxyethoxymethyl, methoxymethyl, methyl, methyl carbonyl, methylsulfonyl, methylthiomethyl, o-nitrobenzyl, p-nitrobenzyl, phenacyl, phenylthiomethyl, 4-picolyl, pivaloyl, propargyl, propyl, tetrafluoro-4-pyridyl, 2-tetrahydropyranyl, p-toluenesulfonyl, 2,2,2-trichloroethyl carbonyl, triethylsilyl, trifluoromethylsulfonyl, trimethylsilyl, 2-(trimethylsilyl)ethoxymethyl and vinyl carbonyl.

It was found, preferably but not exclusively, that favorable results were obtained when said reduction is transfer hydrogenation. In transfer hydrogenation the hydrogen source is not hydrogen gas, as for instance reported in WO 2008/131932. In case of transfer hydrogenation the hydrogen source is, for instance, a salt of formic acid in the presence of a transfer hydrogenation catalyst relying on a completely different reaction mechanism compared to the process as described in WO 2008/131932. Moreover in the biphasic system of the present invention said hydrogen source is present in one of the two phases only whereas the system in WO 2008/131932 is either monophasic and even if some described examples turn out to be biphasic the gaseous hydrogen would be present in all phases making the results of the method of the present invention un-comparable and unpredictable.

In one embodiment the configuration of the product (1) of the method of the present invention preferably is at least 85% R or at least 85% S, more preferably at least 90% R or at least 90% S, still more preferably at least 95% R or at least 95% S and most preferably at least 98% R or at least 98% S.

In a second embodiment the biphasic system of the present invention comprises water and a water-immiscible solvent. Suitable water-immiscible solvents are benzene, butyl acetate, chlorobenzene, chloroform, dichloromethane, ethyl acetate, iso-propyl acetate, toluene, xylene and the like. Preferred water-immiscible solvents leading to very high ee-values are dichloromethane and ethyl acetate. The biphasic system leads to an increase of rate compared to the homogeneous system. This is surprising in the sense that such systems generally are known to be slower in chemical conversions.

In a third embodiment a surfactant is added to the mixture of the method of the present invention. This has a favourable effect on yield and/or ee-value. Preferably the surfactant is a cationic or a zwitterionic surfactant. Examples of suitable surfactants are cetyltrimethylammonium chloride, polyethylene glycol, sodium dodecyl sulphate, tetrabutylammonium bromide, tetrabutylammonium chloride and the like. A particularly suitable surfactant is methyltrioctylammonium chloride. The surfactant may be added prior to the reduction or during the reduction. The amount of surfactant preferably is from 0 to 500 equivalents relative to the amount of catalyst, more preferably from 5 to 100 equivalents, most preferably from 10 to 50 equivalents.

In a fourth embodiment a hydrogen donor is added. Many hydrogen donors are available to the skilled person and examples are formic acid and iso-propanol. We have found salts of formic acid to be particularly suitable. Preferably said formic acid salts are alkaline or alkaline earth salts or amine salts such as ammonium formate or triethylammonium formate. The most preferred salt is sodium formate.

In a fifth embodiment the metal complex catalyst is one based on the metals iridium, rhodium or ruthenium. Particularly suitable catalysts are chloro{[(1R,2R)-(−)-2-amino-1,2-diphenylethyl](4-toluenesulfonyl)amido (also referred as Ts-DPEN)}(mesitylene)ruthenium, chloro{[(1S,2S)-(−)-2-amino-1,2-diphenylethyl](4-toluenesulfonyl)amido}(mesitylene)ruthenium, chloro{[(1R,2R)-(−)-2-amino-1,2-diphenylethyl](pentafluorobenzene-sulfonyl)amido (also referred as Fs-DPEN)}(p-cymene)ruthenium, chloro{[(1R,2R)-(−)-2-amino-1,2-diphenylethyl](methyl-sulfonyl)amido (also referred as Ms-DPEN)}(p-cymene)ruthenium, Cp*-1r(CI)(Ms-DPEN), chloro(mesitylene)-Ru-Ms-DPEN, chloro(m-xylyl)-Ru-Ts-DPEN and chloro(p-xylyl)-Ru-Ts-DPEN, all of which are available from commercial sources and/or may be prepared according to procedures as known to the skilled person.

An advantage of the method of the present invention is that the ratio between substrate and catalyst can be very high so that the catalyst cost and recycling efforts are minimal. For example, the performance of the catalyst in the method of the present invention was found to be 20 times higher than with the system as reported in U.S. Pat. No. 6,148,381. Thus, according to the present invention the ratio between substrate an catalyst may be from 500:1 to 20,000:1, preferably from 750:1 to 10,000:1, more preferably from 1,000:1 to 5,000:1, most preferably from 1,300:1 to 2,100:1.

The compounds of general formula (1) as obtained by the present invention can be converted to montelukast by means of activation of the chiral hydroxyl group followed by displacement and the required modifications to convert group R into R═—C(CH₃)₂OH.

### EXAMPLES

### Analytical Method

The conversion and enantiomeric excess is determined by chiral HPLC using the following conditions:
Column: Chiralcel OD-H (250×4.6 mm ID, 5 μm)
Mobile phase: 95/5 v/v% n-heptane/IPA+0.05% DEA
Flow: 1.5 mL/min.
Detector: UV 254 nm
Inj. Vol.: 5 μL
Time: 33 min
Temperature: 45° C.
Retention times: Ketone (2), R═—C(O)OCH₃: 12.25 min
   S-alcohol (1), R═—C(O)OCH₃: 26.49 min
   R-alcohol (1), R═—C(O)OCH₃: 28.42 min

### Example 1

### Screening of catalysts in the preparation of methyl (E)-2-[3-[3-[2-(7-chloro-2-quinolinyl)ethenyl]phenyl]-3-hydroxypropyl]benzoate ((1), R═—C(O)OCH₃)

Ketone 2 (R═—C(O)OCH₃), sodium formate and a surfactant, methyltrioctylammonium chloride were added as solids in a Schlenk tube in the ratios as given in Table 1. The vessel was placed under inert atmosphere via 3 cycles of vacuum/argon. Degassed water and degassed dichloromethane were added in a ratio of water:dichloromethane=1:2. The catalyst was added in a ratio as given in Table 1 and the reaction mixture was heated to 40° C. At various points in time the conversion and enantiomeric excess was monitored by HPLC analysis. See Table 1 for results.

TABLE 1
Screening of transfer hydrogenation catalysts

| | | | Time | Conversion | e.e. |
|---|---|---|---|---|---|
| Catalyst | Catalyst:Surfactant:2 | HCO₂Na:2 | (h) | (%) | (%) |
| (p-Cymene)-Ru-Fs-DPEN | 1:60:750 | 14 | 1 | 1 | 99 |
| | | | 16 | 24 | 82 |
| (p-Cymene)-Ru-Ms-DPEN | 1:100:1300 | 13 | 1 | 4 | −85 |
| | | | 16 | 49 | −86 |
| | | | 40 | 86 | −86 |
| Cp*-Ir-Ms-DPEN | 1:100:1300 | 13 | 1 | 2 | >−95 |
| | | | 16 | 22 | −90 |
| | | | 40 | 39 | −90 |
| Cp*-Ir-Ts-DPEN | 1:20:300 | 9 | 1 | 1 | −84 |
| | | | 17 | 5 | −79 |
| Cp*-Rh-Ts-DPEN | 1:20:300 | 10 | 1 | 4 | −93 |
| | | | 17 | 4 | −88 |
| (Mesitylene)-Ru-Ts-DPEN | 1:60:1000 | 9 | 1 | 11 | −93 |
| | | | 3 | 27 | −93 |
| | | | 5 | 39 | −93 |
| | | | 7 | 49 | −92 |
| | | | 22 | 90 | −92 |
| (Mesitylene)-Ru-Ms- | 1:20:2000 | 2 | 1.2 | 6 | −93 |
| | | | 3.2 | 15 | −92 |
| | | | 20 | 62 | −92 |
| | | | 28 | 77 | −92 |
| | | | 44 | 94 | −92 |
| (m-Xylyl)-Ru-Ts-DPEN | 1:20:2000 | 2 | 1.3 | 4 | n.d. |
| | | | 3.5 | 10 | n.d. |
| | | | 19.5 | 44 | n.d. |
| | | | 43.5 | 69 | −89 |
| | | | 51.5 | 74 | −88 |
| | | | 67.5 | 82 | −85 |
| (Tolyl)-Ru-Ts-DPEN | 1:20:2000 | 2 | 1.8 | 4 | 66 |
| (S,S)-Ts-DPEN | | | 5 | 10 | 72 |
| | | | 21 | 33 | 73 |
| (Mesitylene)-Ru-Fs-DPEN | 1:30:2000 | 2 | 1.5 | 2 | −59 |
| | | | 5 | 3 | −81 |
| | | | 21 | 9 | −91 |
| | | | 29 | 12 | −91 |
| | | | 45 | 17 | −90 |
| (p-Xylyl)-Ru-Ts-DPEN | 1:35:2100 | 2 | 1.3 | 4 | 81 |
| | | | 3.3 | 11 | 83 |
| | | | 19 | 45 | 83 |
| | | | 27 | 58 | 83 |
| | | | 43 | 75 | 81 |

### Example 2

### Screening of hydrogen donors in the preparation of methyl (E)-2-[3-[3-[2-(7-chloro-2-quinolinyl)ethenyl]phenyl]-3-hydroxypropyl]benzoate ((1), R═—C(O)OCH₃)

Ketone 2 (R═—C(O)OCH₃), a hydrogen donor (see Table 2) and a surfactant, methyltrioctylammonium chloride were added as solids in a Schlenk tube in the ratios as given in Table 2. The vessel was placed under inert atmosphere via 3 cycles of vacuum/argon. Solvent and catalyst were added as outlined in Table 2 and the reaction mixture was heated to 40° C. At various points in time the conversion and enantiomeric excess was monitored by HPLC analysis. See Table 2 for results.

TABLE 2
Screening of hydrogen donors

| | | | | Time | Conversion | e.e. |
|---|---|---|---|---|---|---|
| Catalyst | Catalyst:Surfactant:2 | H₂ donor | Sol. | (h) | (%) | (%) |
| (Mesitylene)-Ru-Ts-DPEN | 1:60:1000 | HCO₂Na | a | 1 | 11 | −93 |
| | | 9 equiv | | 3 | 27 | −93 |
| | | | | 5 | 39 | −93 |
| | | | | 7 | 49 | −92 |
| | | | | 22 | 90 | −92 |
| (Mesitylene)-Ru-Ts-DPEN | 1:70:1000 | HCO₂Na | a | 0.6 | 7 | −90 |
| | | 2 equiv | | 2 | 23 | −93 |
| | | | | 4 | 37 | −93 |
| | | | | 6 | 51 | −93 |
| | | | | 21 | 93 | −93 |
| (Mesitylene)-Ru-Ts-DPEN | 1:70:1000 | HCO₂NH₄ | a | 1 | 4 | n.d. |
| | | 14 equiv | | 18 | 33 | −94 |
| (p-Cymene)-Ru-Ts-DPEN | 1:none:22 | IPA | b | 18 | 54 | −82 |
| Activated with 3 eq. tBuOK | | | | | | |
| (Mesitylene)-Ru-Ts-DPEN | 1:none:1000 | IPA | b | 16 | 11 | −95 |
| Activated with 10 eq. | | | | | | |
| (p-Cymene)-Ru-Ts-DPEN | 1:none:25 | TEAF (2:5) | c | 18 | 36 | >99 |
| | | | | 72 | 100 | 92 |
| (p-Cymene)-Ru-Ts-DPEN | 1:none:20 | TEAF (2:5) | b | 18 | 11 | 88 |
| (p-Cymene)-Ru-Ts-DPEN | 1:none:25 | TEAF (1:1) | c | 18 | 45 | >99 |
| | | | | 72 | 100 | 93 |
| (Mesitylene)-Ru-Ts-DPEN | 1:none:1600 | TEAF (1:1) | a | 18 | 24 | −94 |

a Dichloromethane:water
b Dichloromethane
c Tetrahydrofuran
TEAF: Triethyl amine:HCO₂H

### Example 3

### Screening of solvents in the preparation of methyl (E)-2-[3-[3-[2-(7-chloro-2-quinolinyl)ethenyl]phenyl]-3-hydroxypropyl]benzoate ((1), R═—C(O)OCH₃)

Ketone 2 (R═—C(O)OCH₃), sodium formate and methyltrioctylammonium chloride (surfactant) were added as solids in a Schlenk tube in the ratios as given in Table 3. The vessel was placed under inert atmosphere via 3 cycles of vacuum/argon. (Mesitylene)-Ru-Ts-DPEN as catalyst and solvent were added as outlined in Table 3 and the reaction mixture was heated as indicated. At various points in time the conversion and enantiomeric excess was monitored by HPLC analysis. See Table 3 for results.

TABLE 3
Screening of solvents

| Catalyst: | | | T | Time | Conversion | e.e. |
|---|---|---|---|---|---|---|
| Surfactant:2 | HCO₂Na:2 | Solvent | (° C.) | (h) | (%) | (%) |
| 1:60:1000 | 9 | a | 40 | 1 | 11 | −93 |
| | | | | 3 | 27 | −93 |
| | | | | 5 | 39 | −93 |
| | | | | 7 | 49 | −92 |
| | | | | 22 | 90 | −92 |
| 1:70:1200 | 15 | d | 50 | 0.5 | 6 | −87 |
| | | | | 1 | 10 | −87 |
| | | | | 1.8 | 19 | −87 |
| | | | | 3.8 | 39 | −87 |
| | | | | 6 | 53 | −87 |
| | | | | 20 | 97 | −86 |
| 1:50:700 | 2 | e | 40 | 16 | 16 | −83 |
| | | | | 41 | 41 | −83 |
| | | | | 63 | 63 | −83 |
| 1:60:800 | 2 | f | 40 | 14 | 14 | −93 |
| | | | | 48 | 48 | −93 |
| | | | | 80 | 80 | −93 |

a Dichloromethane:water
d Chlorobenzene:water
e Toluene:water
f Ethyl acetate:water

### Example 4

### Methyl [R-(E)]-2-[3-3-[2-(7-chloro-2-quinolinyl)ethenyl]phenyl]-3-hydroxypropyl]benzoate ((1), R═—C(O)OCH₃); preparation in dichloromethane/water

Substrate to Catalyst Ratio 1000:

Ketone 2 (R═—C(O)OCH₃; 1.18 g, 2.6 mmol), sodium formate (2.65 g, 39 mmol) and methyltrioctylammonium chloride (57 mg, 0.14 mmol) were added as solids in a 500 mL Schlenk tube. The vessel was placed under inert atmosphere via 3 cycles of vacuum/argon. Degassed water (5 mL) and degassed dichloromethane (10 mL) were added. The catalyst, Ru-chloro-(R,R-Ts-DPEN)(mesitylene) (=chloro{R1R,2R)-(−)-2-amino-1,2-diphenylethyl](4-toluenesulfonyl)amido}(mesitylene)ruthenium; 1.6 mg, 0.0026 mmol, substrate to catalyst ratio=1000) was added and the reaction mixture was heated to 40° C. After 20 h, HPLC analysis showed that 94% of the ketone was converted to the alcohol with an enantiomeric excess of 92% (R). The layers were separated and the organic phase was washed with water. Dichloromethane was removed on a rotary evaporator and the residual solids were dissolved in ethanol and crystallized upon addition of a few drops of water. Yield=70%, HPLC purity >99%, ee>99% (R).

Substrate to Catalyst Ratio 1800:

Ketone 2 (R═—C(O)OCH₃; 4.44 g, 9.8 mmol), sodium formate (8.35 g, 123 mmol) and methyltrioctylammonium chloride (235 mg, 0.58 mmol) were added as solids in a 500 mL Schlenk tube. The vessel was placed under inert atmosphere via 3 cycles of vacuum/argon. Degassed water (20 mL) and degassed dichloromethane (32 mL) were added. The catalyst, Ru-chloro-(R,R-Ts-DPEN)(mesitylene) (3.6 mg, 0.0058 mmol, substrate to catalyst ratio=1800) was added and the reaction mixture was heated to 40° C. After 60 h, HPLC analysis showed that 99% of the ketone was converted to the alcohol with an enantiomeric excess of 92% (R). The layers were separated and the organic phase was washed with water. Dichloromethane was removed by rotary evaporator and the residual solids were dissolved in ethanol and crystallized upon addition of a few drops of water. Yield=3.97 g (89%), HPLC purity >99%, ee>99% (R).

### Example 5

### Methyl [R-(E)]-2-[3-[3-[2-(7-chloro-2-quinolinyl)ethenyl]phenyl]-3-hydroxypropyl]benzoate ((1), R═—C(O)OCH₃); preparation in chlorobenzene/water

Ketone 2 (R═—C(O)OCH₃; 1.32 g, 2.71 mmol), sodium formate (2.65 g, 39 mmol) and methyltrioctylammonium chloride (62 mg, 0.153 mmol) were added as solids in a Schlenk tube. The vessel was placed under inert atmosphere via 3 cycles of vacuum/argon. Degassed water (8 mL) and degassed chlorobenzene (8 mL) were added. The catalyst, Ru-chloro-(R,R-Ts-DPEN)(mesitylene) (1.4 mg, 0.0022 mmol, substrate to catalyst ratio=1200) was added and the reaction mixture was heated to 40° C. After 20 h, HPLC analysis showed that 97% of the ketone was converted to the alcohol with an enantiomeric excess of 86% (R).

1. A method for the preparation of an alcohol of general formula (1) 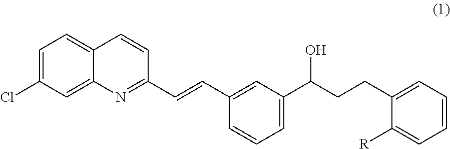
having a configuration of at least 60% R or at least 60% S and wherein substituent R is —C(O)OR₁ or —C(CH₃)₂OR₂ with R₁ is hydrogen or a carboxylic acid protecting group and R₂ is hydrogen or an alcohol protecting group by reduction of a ketone of general formula (2) 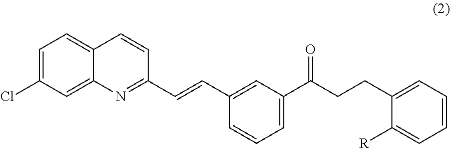
in the presence of a metal complex catalyst, characterized in that said reduction is carried out in a biphasic system.

2. Method according to claim 1 wherein said reduction is transfer hydrogenation.

3. Method according to claim 1 wherein said configuration is at least 95% R or at least 95% S.

4. Method according to claim 1 wherein said biphasic system comprises a water-immiscible solvent and water.

5. Method according to claim 4 wherein said water-immiscible solvent is chlorobenzene and/or dichloromethane and/or ethyl acetate.

6. Method according to claim 1 wherein a surfactant is added prior to said reduction and/or during said reduction.

7. Method according to claim 1 wherein an alkaline or alkaline earth metal salt of formic acid is added prior to said reduction and/or during said reduction.

8. Method according to claim 1 wherein said metal complex catalyst is an iridium complex catalyst, a rhodium complex catalyst or a ruthenium complex catalyst.

9. Method according to claim 8 wherein said ruthenium complex catalyst is chloro{[(1R,2R)-(−)-2-amino-1,2-diphenylethyl](4-toluenesulfonyl)amido}(mesitylene)ruthenium or chloro{[(1S,2S)-(−)-2-amino-1,2-diphenylethyl](4-toluenesulfonyl)amido}(mesitylene)ruthenium or chloro{R1R,2R)-(−)-2-amino-1,2-diphenylethyl](pentafluorobenzenesulfonyl)amido}(p-cymene)ruthenium or chloro{[(1R,2R)-(−)-2-amino-1,2-diphenylethyl](methyl-sulfonyl)amido}(p-cymene)ruthenium.

10. Method according to claim 1 for preparing an optically active alcohol of formula (1) as an intermediate in the preparation of 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropaneacetic acid or a pharmaceutically acceptable salt thereof.
